(19) United States
(12) Patent Application Publication
(10) Pub. No.: US 2023/0087154 A1
(43) Pub. Date: Mar. 23, 2023

(54) MEDICAL SYRINGE AND PISTON STOPPERS FOR A MEDICAL SYRINGE

(71) Applicant: Fresenius Kabi Deutschland GmbH, Bad Homburg (DE)

(72) Inventors: Torsten Brandenburger, Reichelsheim (DE); Michael Schönhofen, Saarlouis (DE)

(21) Appl. No.: 17/759,761

(22) PCT Filed: Apr. 22, 2021
(86) PCT No.: PCT/EP2021/060472

§ 371 Date: Jul. 29, 2022

(30) Foreign Application Priority Data
Apr. 24, 2020 (EP) 20171391

(51) Int. Cl. A61M 5/315

(52) U.S. Cl. CPC A61M 5/31513; A61M 2207/10

(57) ABSTRACT

The invention relates to a piston stopper (10) for a medical syringe (1) wherein the piston stopper (10) has an end wall (11), a lateral wall (12) and an internal thread (16) for securing a piston rod (7). The piston stopper (10) comprises a main body (10a) which made of a cycloolefin, in particular a cycloloefin copolymer or a cycloolefin polymer. A plurality of sealing rings (13a-13d) that are made from a plastic material extend around the main body of the piston (10) and are materially bonded to the main body of the piston (10a). The invention furthermore relates to a medical syringe (1) and to a method for the production thereof.

### FIELD OF THE INVENTION

The invention relates to a medical syringe and a plunger stopper for a medical syringe. The invention in particular relates to a medical syringe packaged in a sterile manner which is pre-filled with a solution or emulsion containing an active ingredient.

### BACKGROUND OF THE INVENTION

Pre-filled medical syringes made of plastic are known. Such a medical syringe consists of a syringe body with a nozzle for delivering the medical fluid and a plunger with a plunger rod.

In the case of pre-filled medical syringes, i.e. single-use syringes, which are delivered already filled with a fluid containing an active ingredient, the requirements for the materials used are high. The syringe must be sealed tight, the materials used should be autoclavable and there should be no interactions between the materials used and the filled fluid even in the case of extended periods of contact with the medical fluid.

Thus, leaching of the used plastics in the near-surface region due to the medical fluid and the associated migration of components of the plastic into the medical fluid or of components of the medical fluid into the plastic should in particular be avoided as far as possible.

In the case of known medical syringes, the plunger and the plunger rod usually do not consist of one piece, but rather a plunger stopper made of an elastic material is used, which is then connected to the plunger rod.

The selection of suitable elastic materials is limited, depending on the active ingredient for which the syringe is used.

In addition to interactions with the filled medical fluid that are as low as possible, the mechanical properties of the plunger stopper also play an important role.

Thus, due to different frictional forces around the circumference of the plunger, tilting of the plunger stopper may result, whereby the necessary force for pushing further is rapidly increased.

In order to provide a syringe with improved seal-tightness and low interaction in terms of the filled fluid, the published patent application WO 2014/001386 A1 provides that the plunger stopper consists of two different materials. Thus, this document shows a main body of the plunger, which consists in particular of a cycloolefin copolymer, and has grooves, into which are pressed two O-rings made of bromobutyl and acting as sealing rings.

This configuration, however, entails quite a high assembly effort and, due to the pressed O-rings, which can only deflect to a limited extent, is not very tolerant with respect to dimensional deviations of the syringe body.

### OBJECT OF THE INVENTION

Therefore, the object underlying the invention is to provide a plunger stopper or a syringe with a plunger stopper, which is or are further improved in terms of seal-tightness, necessary pushing force and/or possible interactions with the filled medical fluid.

### SUMMARY OF THE INVENTION

The object of the invention is already achieved by a plunger stopper according to the independent claims and by a medical syringe equipped with the plunger stopper and by a method for manufacturing a medical syringe.

Preferred embodiments and further developments of the invention can be inferred from the subject matter of the dependent claims, the description and the drawings.

According to a first aspect, the invention relates to a plunger stopper for a medical syringe, wherein the plunger stopper has an end wall, a lateral wall and an internal thread for securing a plunger rod, wherein the plunger stopper comprises a main body, which comprises a cycloolefin, in particular a cycloolefin copolymer, a cycloolefin polymer and/or a crystal clear polymer, wherein a plurality of sealing rings made of an elastic material extend around the main body of the plunger and are connected to the main body of the plunger in a materially-bonded manner.

A cycloolefin in the context of the invention is understood as all cycloolefin copolymers which are obtained by catalyzed copolymerization of cycloolefins.

In the context of the invention, cycloolefins are also understood as materials, which are obtained by a ring-opening salt metathesis. These are, strictly speaking, not designated as cycloolefin copolymers, but as cycloolefin polymers.

The cycloolefin can also be formed as a crystal clear polymer.

The main body of the plunger preferably consists to at least 50% by weight, particularly preferably to at least 90% by weight, of a cycloolefin.

Such materials are amorphous, transparent and, to a notably lesser extent than other plastics, enter into interactions with the filled medical fluid, in particular with an aqueous medical fluid.

Due to the materially-bonded connection between the main body of the plunger and the sealing rings, they do not have to be under pre-tension, preferably the sealing rings are not under pre-tension.

The elasticity and the cross-section of the respective sealing ring can therefore be selected substantially completely independently of the main body of the plunger.

The main body of the plunger is, according to the invention, provided with at least two sealing rings. According to a further development of the invention, three or four sealing rings can also be provided.

According to one embodiment of the invention, all sealing rings have the same cross-section.

According to one embodiment of the invention, the main body of the plunger is overmolded with the sealing rings. This has the advantage that a materially-bonded connection can be provided in a simple manner, in particular without further additives.

According to one embodiment of the invention, the sealing rings have at least in sections a rounded shape, in particular the shape of a circular segment. In particular, the sealing rings can have the shape of a semi-circle.

According to a preferred embodiment, the cross-section of a sealing ring is composed of a circular segment and a polygon, in particular a rectangular base. The polygonal base can thus be arranged in a corresponding groove of the main body of the plunger, whereas the circular segment protrudes radially from the main body of the plunger and can be compressed when inserted into the syringe body.

In the case of a further development of the invention, the distally first sealing ring is covered at least partially distally by a circumferential web of the main body of the plunger. The web enlarges the end wall to form a cover which covers the distally forward sealing ring. The web preferably directly adjoins the distally first sealing ring.

The circumferential web forms the largest diameter of the main body of the plunger here, i.e. the diameter of the main body of the plunger is smaller behind the web, in particular over the entire length of the main body of the plunger.

Due to the circumferential web, at least one part of the sealing ring is covered, whereby an interaction of the material of the sealing ring with the medical fluid is further reduced since the web is formed of a cycloolefin.

It is in particular provided that when the syringe is assembled, the web can rest or rests on the inner wall of the main body of the plunger such that the plunger stopper can still be moved or displaced in the syringe body. The outer diameter of the web corresponds substantially to an inner diameter of the syringe body or is somewhat smaller in one configuration of the invention.

The distally foremost sealing ring preferably protrudes in the unassembled state beyond the web at the edge. It is in particular provided that the sealing ring protrudes 0.05 to 0.5 mm, preferably 0.08 to 0.2 mm, beyond the edge of the web. During assembly, the sealing ring is compressed in such manner that the web now rests on the inner wall of the syringe body.

Due to the harder material of the main body of the plunger, the web acts only to a limited extent as a seal. The seal-tightness of the syringe is, however, ensured by the subsequent sealing rings.

The distally first sealing ring preferably directly adjoins the web. The possible volume between web and sealing ring is thus kept low. In particular, this is largely filled by the deflecting sealing ring.

The web preferably has a height of less than 1 mm, particularly preferably of less than 0.5 mm. The web is thus formed as a circumferential rib.

The circumferential web can also be rounded at the edge in order to facilitate the syringe body sliding along the lateral wall.

In one embodiment of the invention, the end wall of the plunger stopper is conical on the outer side and on the inner side in the distal direction.

The distal outer cone preferably corresponds substantially to a cone of the main body of the syringe so that it is ensured that the syringe can be virtually completely emptied.

A conical shape on the inner side, i.e. the proximal side of the end wall, can entail advantages when guiding the plunger stopper. In particular, a correspondingly conical plunger rod can have a conical end, whereby the plunger stopper is centered when pushed.

In the case of one embodiment of the invention, the sealing rings consist of a butyl rubber, in particular a halogenated butyl rubber, such as e.g. a chlorobutyl and/or bromobutyl. As described at the outset, these materials have the advantage of a low interaction with medical fluids and in particular hardly tend to leach.

In particular, in the case of the embodiment in which the distally foremost sealing ring is covered by the web, the sealing rings can, however, also consist of another plastic.

Silicone or polypropylene come into consideration in particular as other plastic materials.

In the case of a further embodiment, the internal thread of the plunger stopper is formed as a double trapezoidal thread.

Such a thread distributes the acting forces over a large area.

The plunger stopper can also have a coating. In particular, the sealing rings and/or the main body of the plunger can be provided with a coating. This can for example be a coating made of a polytetrafluoroethylene which can serve as a migration barrier and also an anti-friction layer.

In the case of one embodiment of the invention, the ratio of a wall thickness of the lateral wall to a diameter of the plunger stopper is more than 0.03, preferably more than 0.05, particularly preferably more than 0.06 and/or less than 0.2, preferably less than 0.1, particularly preferably less than 0.08. It has been found that this ratio can achieve a particularly good guidance of the plunger stopper.

According to one embodiment of the invention, the sealing rings have a height of more than 0.5 mm, preferably more than 1.0 mm, particularly preferably more than 2 mm, and/or less than 5 mm, and/or preferably less than 3 mm. The height of the sealing ring is understood as the extension proceeding from a contact surface of the sealing ring to the main body of the plunger in the radial direction.

A part of the sealing ring can thereby sit in a groove of the main body of the plunger, as described above.

The end wall is preferably a truncated cone shape. The end wall can have a proximal tip angle on the outside which corresponds in particular to the distal tip angle of the end wall, i.e. on the inside. The end wall is thus roof-shaped in an axial cross-section.

The proximal and/or the distal tip angles can in particular be over 100°, preferably over 120°, particularly preferably over 140° and/or under 170°, preferably under 160°, particularly preferably under 155°.

The thread, in particular the trapezoidal thread, is, in one embodiment, formed in such manner that the ratio of a tooth height of the internal thread to a tooth diameter of the plunger stopper is over 0.04, particularly preferably over 0.05 and/or under 0.15.

The diameter of the plunger stopper is, according to one embodiment of the invention, between 15 mm and 30 mm.

The wall thickness of the end wall is, in one embodiment of the invention, 1 to 2 times, preferably 1.2 to 1.6 times, the wall thickness of the lateral wall.

The plunger stopper is partially thickened on the lateral wall since the teeth of the internal thread protrude from the lateral wall on the inside. Due to a corresponding dimensioning of the end wall, there are similar size proportions on the end face and in terms of the lateral wall. This facilitates the manufacture of the plunger stopper. In particular, tensions of the material during injection molding are reduced as a result, e.g. due to differing cooling.

The pitch of the internal thread is, in the case of one embodiment of the invention, between 0.4 and 0.8 mm, preferably between 0.5 and 0.7 mm. The plunger stopper can have a total length, including the conical end wall, of between 8 mm and 20 mm, preferably of between 14 mm and 16 mm.

The invention also relates to a syringe, which is equipped with a plunger stopper as previously described.

The syringe body can thereby be provided from the same material as the main body of the plunger.

It is in particular provided that the syringe body and the main body of the plunger consist of a cycloolefin, in particular of the same cycloolefin. This reduces the number of different materials and therefore facilitates the restriction of possible interactions with the medical fluid.

As previously described, the circumferential web can in particular rest on the inner wall of the syringe body.

According to a preferred embodiment, the syringe comprises a plunger rod, in particular a conical distal end, which, when the plunger rod is connected to the plunger stopper, rests on a preferably conical inner side of the end wall of the plunger stopper. When the plunger is pushed, it is thus centered and therefore tends to tilt to a lesser extent.

The syringe is in particular formed as a pre-filled syringe, which for example is filled with a medical fluid containing a medication.

According to one embodiment, the medical fluid is a pharmaceutical fluid that is sensitive to oxygen, for example a pharmaceutical emulsion. According to one embodiment, the pharmaceutical fluid is or comprises propofol, in particular a propofol emulsion. Propofol is designated by the chemical name 2,6-diisopropylphenol (IUAPC).

The distal end of the syringe, i.e. the nozzle, is sealed with a stopper.

In addition to the nozzle, the syringe can comprise a connection piece, in particular a threaded connection. In particular, the syringe can comprise a male Luer lock connection by means of which the syringe can for example be connected to a transfer set to apply the medical fluid.

The syringe is preferably autoclaved and in particular arranged in an outer package impermeable to oxygen, such as for example a foil package.

According to a further aspect, the invention relates to a syringe, in particular a syringe with one or a plurality of features described above.

According to this aspect of the invention, the syringe comprises a syringe body and a plunger stopper inserted into the syringe body, which comprises a main body of the plunger and at least one sealing ring, wherein the sealing ring is covered at least partially distally by a circumferential web of the main body of the plunger, wherein the web protrudes radially from the main body of the plunger and the main body of the plunger has a smaller outer diameter than the circumferential web proximally behind the at least one sealing ring.

As mentioned above, the circumferential web forms a distal cover, which reduces the contact of the sealing ring with the medical fluid.

The invention also relates to a method for manufacturing a syringe, in particular a syringe with one or a plurality of features described above, comprising the following steps:

Injection-molding a syringe body which is formed at least in sections of a first material, Injection-molding a main body of the plunger from the first material, Injecting at least one sealing ring on the main body of the plunger to form a plunger stopper, Inserting the plunger stopper into the syringe body.

The syringe body can be manufactured from a plurality of different materials. However, at least the inner wall of the syringe body is preferably formed of the first material. The first material is in particular a cycloolefin.

Since the inner wall of the syringe body and of the plunger consist of the same material, possible interactions with a medical fluid can be reduced.

### BRIEF DESCRIPTION OF THE DRAWINGS

The subject matter of the invention will be explained in more detail below on the basis of the drawings FIG. 1 to FIG. 5 with reference to exemplary embodiments.

FIG. 1 is a sectioned view of an exemplary embodiment of a syringe according to the invention.

FIG. 2 is a side view of the plunger stopper used for the syringe.

FIG. 3 is a sectioned view along the line A-A of FIG. 2.

FIG. 4 is a sectioned view of a further exemplary embodiment of a plunger stopper.

FIG. 5 shows the method steps according to one exemplary embodiment of the invention for manufacturing a syringe.

### DETAILED DESCRIPTION OF THE DRAWINGS

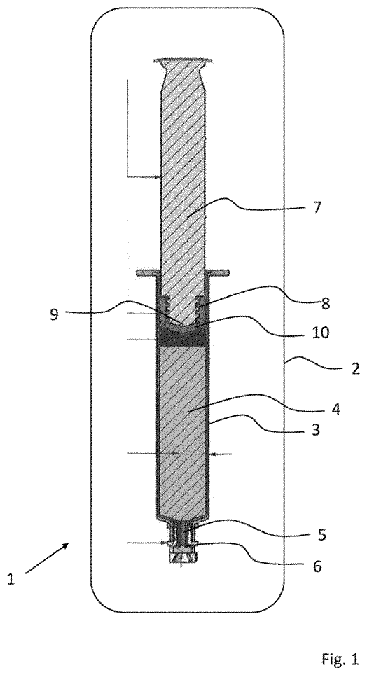

FIG. 1 shows, in a side view, a medical syringe 1 which is equipped with a plunger stopper 10 according to the invention.

The syringe 1 is arranged here in a schematically represented outer package 2. The outer package 2 is formed as a tear-open foil package and the syringe 1 has been autoclaved in particular in the outer package 2.

The syringe 1 comprises a syringe body 3, which provides a volume which is pre-filled with a medical fluid 4. The nozzle 5 of the syringe 1 is sealed with a stopper 6.

In addition to the medical fluid 4, the inner volume of the syringe 1 can contain a gas, in particular nitrogen, which is free of or reduces oxygen, in order to avoid deformations of the syringe body 3 in the case of pressure fluctuations.

In order to expel the medical fluid 4 after removing the stopper 6, the syringe 1 comprises a plunger rod 7 with a proximal gripping end, which is pre-assembled here by way of example, said gripping end being connected to the plunger stopper 10 via its distal end. To this end, the plunger rod 7 comprises an external thread 8, which is screwed into the internal thread 16 of the plunger stopper 10. The distal end 9 of the plunger rod 7 is conical, in this exemplary embodiment cone-shaped, and rests on the end wall 11 of the plunger stopper 10 correspondingly formed on the inside.

The plunger rod 7 may also not be pre-assembled, for example provided lying next to the syringe body 3 in the outer package 2 (not represented here).

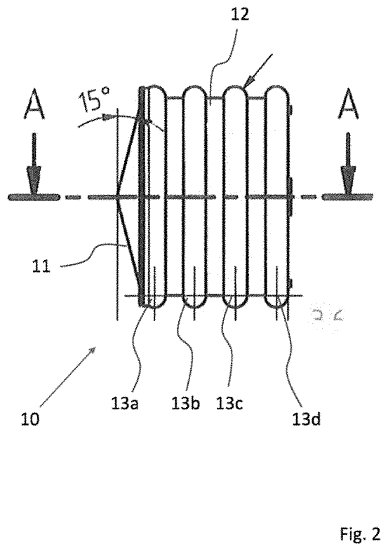

FIG. 2 shows, in a side view, an exemplary embodiment of a plunger stopper 10 according to the invention.

The plunger stopper 10 comprises a conical, in particular truncated cone-shaped end wall 11 and a substantially circular-cylindrical lateral wall 12.

In the exemplary embodiment represented here, the plunger stopper 10 comprises in total four sealing rings 13a to 13d which project or protrude radially from the lateral wall 12.

A distally first sealing ring 13a directly adjoins the end wall 11 in an axial direction. A proximally first sealing ring 13d forms the proximal end of the plunger stopper 10.

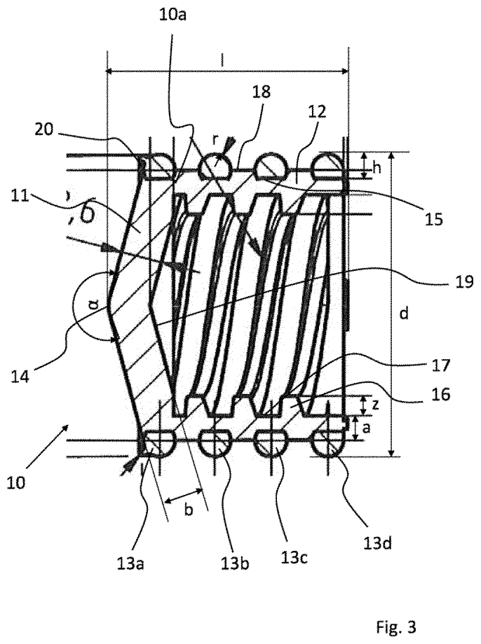

As represented in the sectioned view according to FIG. 3, the plunger stopper 10 is formed by a main body of the plunger 10a and the injected sealing rings 13a to 13d.

The main body of the plunger 10a is formed of a cycloolefin, whereas the sealing rings 13a to 13d are formed of a different elastic material, such as for example a halogenated butyl rubber.

The sealing rings 13a to 13d sit in grooves 15 of the lateral wall 12 of the main body of the plunger 10a in the exemplary embodiment represented here.

An intermediate region, which runs straight, in particular is formed circular-cylindrically in the sectioned view, and which forms a circumferential web 18 in each case, is present between the sealing rings 13a to 13d.

In this exemplary embodiment, the sealing rings 13a to 13d have in their base region substantially the same cross-section. Said cross-section is formed of a circular segment-shaped, in particular semi-circular section, which merges into a polygonal, in particular substantially rectangular base section, which sits in a groove 15 in each case.

Thus, an optimal connection is provided between the sealing rings 13a to 13d and the main body of the plunger 10a.

In this exemplary embodiment, the inner radius r of the circular segment-shaped section is 0.8 to 1.2 mm. The sealing rings 13a to 13d have a total height h of between 1.0 to 1.5 mm.

By definition, the wall thickness a of the lateral wall 12 is determined from the distance of the inner wall to the circumferential web 18 between the sealing rings 13a to 13d. Thus, the wall thickness a is not reduced by the grooves 15 by definition. In this exemplary embodiment, the wall thickness a is between 1.3 and 1.8 mm.

The teeth of the internal thread 16 formed as a double trapezoidal thread protrude from the inner side of the lateral wall 12. The tooth height z between the tooth base 17 and the tooth tip can in particular be 1.2 to 1.7 mm. The internal thread 16 extends here substantially beyond the total length of the lateral wall 12.

The end wall 11 is a truncated cone shape and has a tip 14 with the tip angle α of 140 to 160°. The inner side 19 of the end wall 11, i.e. the distal side is formed correspondingly conical here, such that a uniform wall thickness b is formed. In this exemplary embodiment, the wall thickness b of the end wall 11 is 1.2 to 1.6 times the wall thickness a of the lateral wall.

The plunger stopper 10 has a total length 1 of 14 to 16 mm. In this exemplary embodiment, the diameter d is between 18 and 20 mm. The plunger stopper represented here is formed for syringes 1 with a fill volume of roughly 20 ml.

In order to reduce the contact of the filled medical fluid with the sealing rings 13a to 13d, the main body of the plunger 10a comprises a distally circumferential web 20, which directly adjoins the upper side of the end wall 11.

The web 20 protrudes radially outwards in a plate-shaped manner and is aligned perpendicular to the central axis of the plunger stopper 10. The length in the axial direction of the web 20 is less than 0.5 mm.

The web 20 directly adjoins the distally foremost sealing ring 13a. Thus, the web 20 forms a cover, which covers the sealing ring 13a in the inserted state.

The sealing ring 13a protrudes between 0.08 and 0.15 mm beyond the outer edge of the web 20 in the non-inserted state represented here.

When the plunger stopper 10 is inserted into the syringe body 3, the sealing rings 13a to 13d are compressed and the web 20 preferably rests directly on the inner wall of the syringe body 3 in such manner that the plunger stopper 10 can be pushed in the syringe body 3.

In order to achieve improved sliding properties, the web 20 is rounded at the edges, in particular with an edge radius of between 0.2 and 0.4 mm.

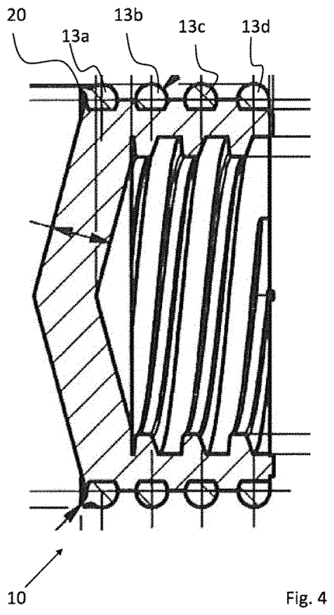

FIG. 4 shows an exemplary embodiment of a plunger stopper 10, which is formed for larger syringes 1, preferably with an inner volume of 50 ml. The sealing rings 13a to 13d and the edge-side, circumferential web 20 are formed corresponding to FIG. 3.

The diameter d of the plunger stopper is between 25 and 28 mm. The lateral wall 12 and/or the end wall 11 are formed thicker roughly corresponding to the diameter. The length 1 is, in contrast, substantially identical to the plunger stopper 10 represented in FIG. 3.

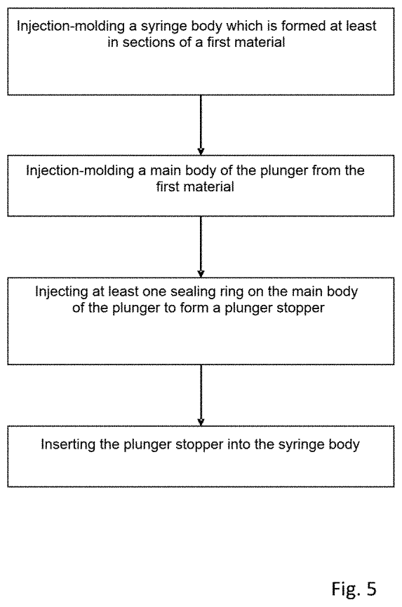

FIG. 5 shows, in a flow diagram, the steps of a method according to the invention for manufacturing a medical syringe 1 according to one exemplary embodiment of the invention.

First, a syringe body 3, which is formed at least in sections of a first material, is injection-molded by means of an injection-molding process.

The first material preferably forms at least the inner wall of the syringe body 3. This may in particular be a cycloolefin. Then, a main body of the plunger 10a is injection-molded from the same material, in particular also from a cycloolefin. At least one sealing ring 13a-13d, preferably a plurality of sealing rings 13a-13d, is injected on the main body of the plunger 10a to form a plunger stopper 10. The plunger stopper 10 can now be inserted into the syringe body 3. The syringe 1 is preferably filled and sealed beforehand with a stopper 6. The syringe 1 can then be inserted into an outer package 2. The syringe 1 is autoclaved in the outer package 2 and is now also provided as a sterile finished syringe for extended storage.

A syringe 1 suitable for finished syringes can be provided with improved properties by way of the invention.

### LIST OF REFERENCE NUMERALS

α Tip angle
a Thickness of the lateral wall
b Thickness of the end wall
d Diameter of the plunger stopper
h Height of the sealing rings
1 Length of the plunger stopper
r Inner radius of a sealing ring
z Tooth height of the internal thread
1 Syringe
2 Outer package
3 Syringe body
4 Medical fluid
5 Nozzle
6 Stopper
7 Plunger rod
8 External thread of the plunger rod
9 Distal end of the plunger rod
10 Plunger stopper
10a Main body of the plunger
11 End wall
12 Lateral wall
13a-13d Sealing ring
14 Tip
15 Groove
16 Internal thread
17 Tooth base
18 Circumferential web of the lateral wall
19 Inner side of the end wall
20 Web

1. A plunger stopper for a medical syringe the plunger stopper comprising:
an end wall, a lateral wall and an internal thread for securing a plunger rod, and
a main body, which comprises a cycloolefin, with a plurality of sealing rings made of an elastic material extending around the main body and connected in a materially-bonded manner to the main body.

2. The plunger stopper according to claim 1, wherein the main body is overmolded with the sealing rings.

3. The plunger stopper according to claim 2, wherein the sealing rings are formed in the cross-section of a circular segment and a polygonal base.

4. The plunger stopper according to claim 1, wherein the sealing rings sit in circumferential grooves of the main body.

5. The plunger stopper according to claim 1, wherein the plurality of sealing rings comprises at least three sealing rings.

6. The plunger stopper according to claim 1, wherein a distally first sealing ring is covered at least partially distally by a circumferential web of the main body.

7. The plunger stopper according to claim 6, wherein:
the end wall of the main body of the plunger merges into the circumferential web at an edge and/or
circumferential web has a height of less than 1 mm and/or
the circumferential web is rounded at the edge and/or
the distally first sealing ring protrudes in a plan view of the distal side of the plunger stopper between 0.05 and 0.5 mm beyond an outer edge of the web and/or
the circumferential web rests on an inner wall of the syringe body.

8. The plunger stopper according to claim 1, wherein the cycloolefin comprises at least one cycloolefin selected from a group consisting of cycloolefin copolymer, cycloolefin polymer and crystal clear polymer.

9. The plunger stopper according to claim 1, wherein the plurality of sealing rings comprise at least one plastic selected from a group consisting of butyl rubber, silicone and polypropylene.

10. The plunger stopper according to claim 1, wherein a proximal tip angle of the end wall corresponds substantially to a distal tip angle of the end wall.

11. A syringe with a plunger stopper according to claim 1.

12. A syringe with a plunger stopper according to claim 6, wherein the circumferential web rests on the an inner wall of the syringe body.

13. The syringe according to claim 11, wherein the syringe has a plunger rod with a conical distal end, which, with the plunger rod is connected to the plunger stopper, rests on a conical inner side of the end wall of the plunger stopper.

14. The syringe according to claim 11, comprising a syringe body and the plunger stopper inserted into the syringe body, wherein the plurality of sealing rings comprises a first sealing ring is covered at least partially distally by a circumferential web of the main body of the plunger, wherein the web protrudes radially from the main body and the main body has a smaller outer diameter than the circumferential web proximally behind the first sealing ring.

15. A method for manufacturing a syringe according to claim 11, comprising the steps:
injection-molding a syringe body which is formed at least in sections of a first material,
injection-molding the main body of the plunger stopper from the first material,
injecting the plurality of sealing rings on the main body to form the plunger stopper,
inserting the plunger stopper into the syringe body.

16. The plunger stopper according to claim 3, wherein the polygonal base comprises a rectangular base.

17. The plunger stopper according to claim 5, wherein the plurality of sealing rings comprises at least four sealing rings.

18. The plunger stopper according to claim 7, wherein the circumferential web has a height of less than 0.5 mm.

19. The plunger stopper according to claim 7, wherein the distally first sealing ring protrudes in a plan view of the distal side of the plunger stopper between 0.08 and 0.2 mm beyond an outer edge of the web.

20. The plunger stopper according to claim 9, wherein the butyl rubber comprises a halogenated butyl rubber comprising chlorobutyl or bromobutyl.
